(19) United States
(12) Patent Application Publication
(10) Pub. No.: US 2013/0092180 A1
(43) Pub. Date: Apr. 18, 2013

(54) Heel Saver

(71) Applicant: Josephine Borquez Garcia, Tucson, AZ (US)

(72) Inventor: Josephine Borquez Garcia, Tucson, AZ (US)

(21) Appl. No.: 13/274,956

(22) Filed: Oct. 17, 2011

(51) Int. Cl. A61F 13/06

(52) U.S. Cl. 128/893

(57) ABSTRACT

By placing material made of foam or gel between the heel of the shoes and the heel of your foot, clipped onto the back by a thin metal strip and also held secure by adhesive. This creation is not only is comfortable, but adds style to your outfit or look. The thin metal attachment which is held in place by two equal pieces of material, center, then folded outward to allow the attachment of embellishments, adds not only comfort, but also opens up fashion ideas and options. Presently there is no item that cannot be found in the market today that is a solution to this problem, which cover the top rim of the shoe.

### BACKGROUND OF THE INVENTION

A void existed in the market that needed to be filled with a preventative solution for an extremely common problem affecting the health of the human foot. Nothing is worse than a blister -- those red, burning sores, puffed up and filled with fluid, that appear when shoes rub sensitive skin the wrong way. New or ill-fitting shoes are most often the cause of blisters on the heel of the foot, which occur whenever you're “breaking in” new shoes. Currently there are no options available for consumers to purchase on the market to aid in the prevention of this painful foot aliment. In turn, people have resorted to the only sensible solution imaginable, which is not at all practical. Consumers cover the heel of the foot with a Band-Aid to cover the red-sore area where they sense a blister developing. Unfortunately, almost immediately, the band-aid losses its adhesive properties especially after continuous use, to which the band-aid dislodges and falls off. Thus, beginning an inconvenient cycle of constantly re-adjusting a messy and unattractive band-aid or replacement of such, leaving the blistered area raw again, from this need an idea germinated and grew into the inspiration for the Heel Saver. The Heel Saver is an adhesive patted clip that provides a cushioned barrier between the back of the foot and the back of the shoe, providing the stability and comfort essential to protecting the sensitive skin around the heel of the foot from irritation.

### SUMMARY OF INVENTION

Heel Saver is an attachment for the back of a shoe that helps to prevent the straps or the heel rim of the shoe from rubbing against the back of the foot. The attachment assists in relieving the discomfort caused from the friction. This product provides a decorative embellishment for footwear so that it is not only comfortable, but also fashionable. This product is ideal for use by women and can be attached to stilettos, high-heeled sandals, and other types of shoes. While, solving the following problems, many women wear shoes that are uncomfortable and often times find themselves wearing the shoes for prolonged periods of time. Straps or the heel rim may rub against the foot, causing sores and irritation. Many women find it unbearable to continue wearing these shoes. A more comfortable alternative is needed. Heel Saver accomplished this in its built. Heel Saver is an attachment that can be clipped onto the back of a shoe. The attachment may be made from a variety of materials, including cotton, rubber, foam and gel. The foam and/or gel material rests against the foot, while the decorative portion of the attachment sits outside the shoe so that it is visible. The clipping and holding mechanism is between the two sides of foam in addition to the adhesive surface of the self adhesive foam side. A wide variety of embellishments may be available, such as flowers, diamonds, pearls, and other stones. The shape and size of the attachment may vary based upon the size and model of the footwear. Specifications may vary.

### BRIEF DESCRIPTION OF FIGURES

FIG. 1 is an aerial view of the inventions parts

FIG. 2 is a step by step instructional process displayed for assembling of metal strip covered by form and wrapped by color ribbon

FIG. 3 is a step by step instructional processed displayed for assembling of pocket to insert Part made in FIG. 2

FIG. 4 is a display of assembling for part created in FIG. 2 & FIG. 3 joined together

FIG. 5 is the final proto-type for the “Heel Saver”

### DETAILED DESCRIPTION

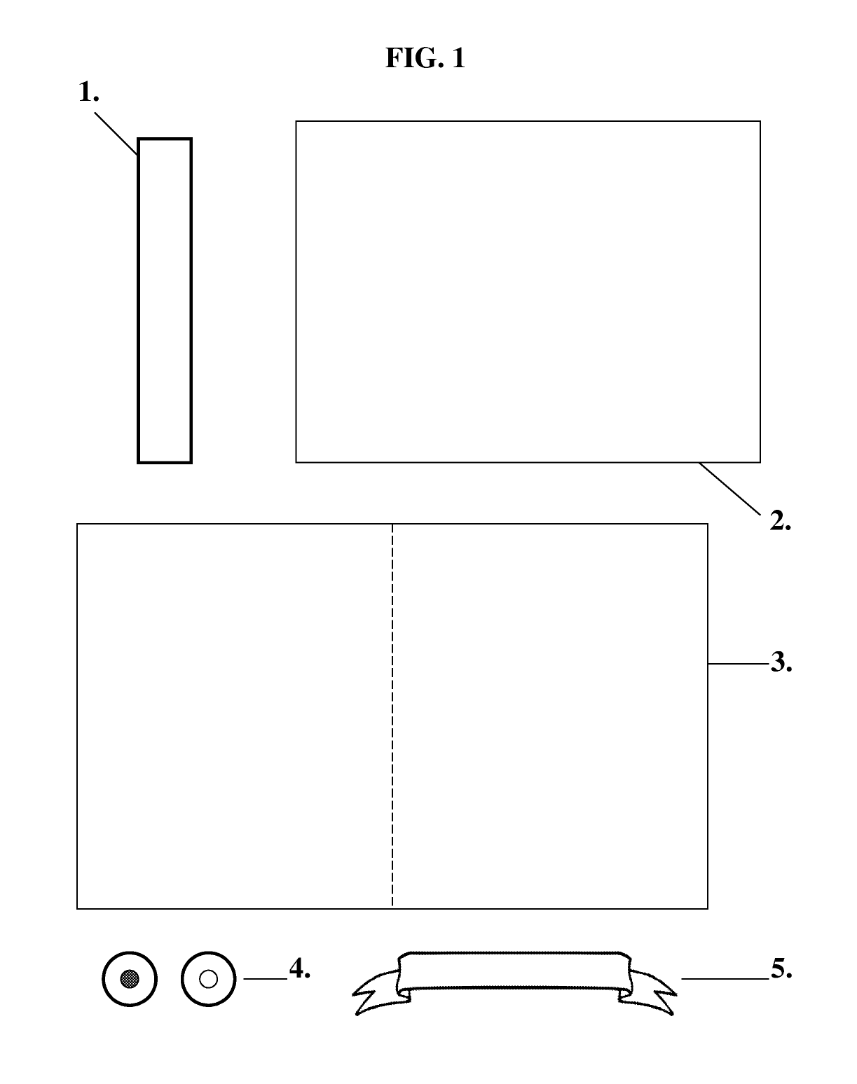

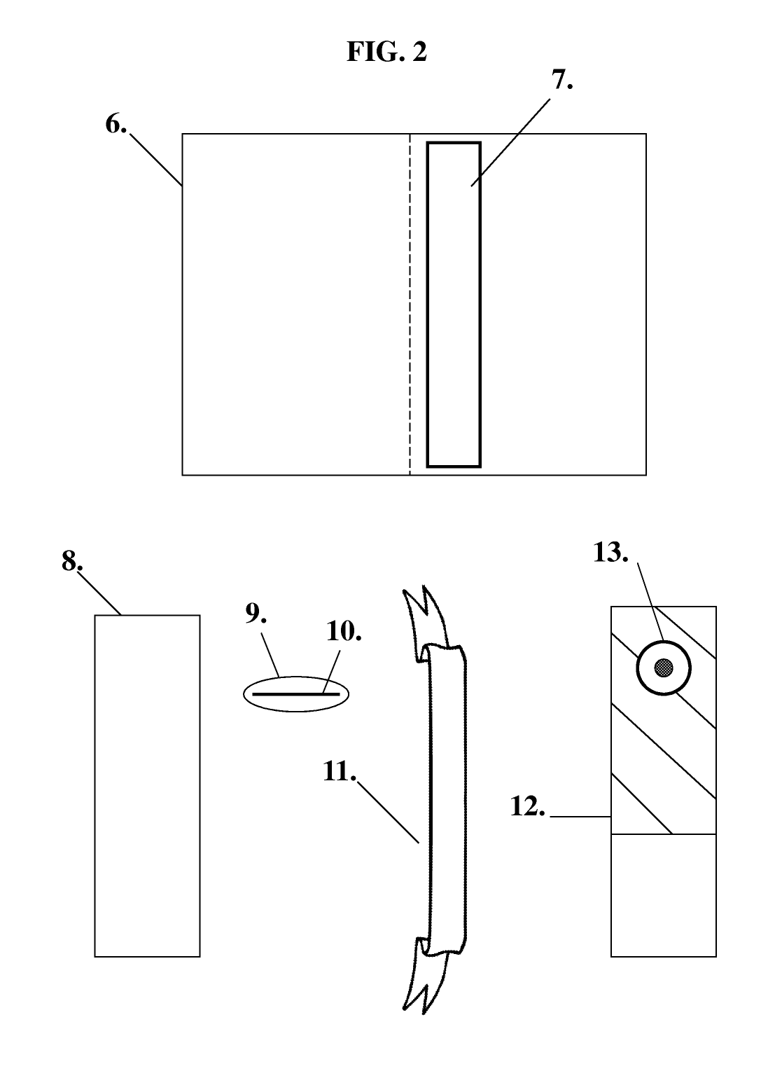

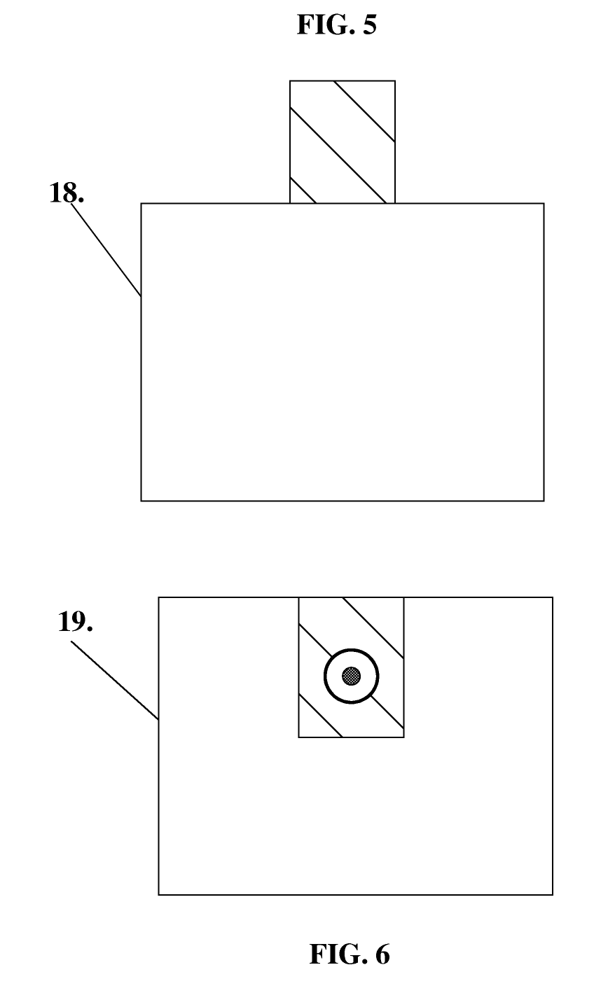

To begin the assembly, take metal strip #1 and off-center it atop self adhesive foam piece #2, then proceed to take the left side and fold it flush to the opposite edge, covering the metal strip entirely, trim extra material. You will now have an item as illustrated by FIG. 2 #8. Now take fabric ribbon, folded length wise and attach Metal Snap Button as presented in FIG. 1 #4 about 1″ from left side. Then insert the tip of the prepared strip and place next to Snap Button and begin wrapping tightly around prepared strip. For now set aside this newly formed piece. Now take one sided self adhesive foam piece as described in FIG. 1 #3 and cut equally into two (2) pieces. Lay down 1 piece with adhesive part facing down and center the strip with the Metal Snap Button facing down, lay on top the other foam piece with adhesive side facing down and seal together. Now take newly form part as illustrated in FIG. 5 and fold the strip over. You now have a “Heel Saver” which can be inserted into the back of the shoe and secured into place with the fold portion of the strip, in addition to the adhesive from foam piece. Attachments for decoration can now be attached to the metal snap button, however; this will be left open to design construction which will allow for the fabrication to allow Gluing, Sewing, and welding or all to completing the look. These attachments will be available in a variety of shapes, designs, styles of various embellishments, such as but not limited to: Stones, Crystals, Flowers, and Various shape forms.

It will be understood that the invention is not to be limited to the details given herein but that it may be modified within the scope of the appended claims. In addition, it will be understood that the specifications and dimensions will vary depending on the size of the wearer, i.e . . . . adult and/or children sizes, in addition to Small Size, Medium Size, Large Size, etc . . .

### Key to Numbering

FIG. 1
1 3½″×¾″×1 cm Metal Strip
2 4½″×2″ One Sided Self Adhesive Foam Piece
3 2½″×3″ One sided Self Adhesive Foam Piece
4 Metal Snap Button
5 7″×1½″ Fabric Ribbon

FIG. 2
6 4½″×2″ One sided Self Adhesive Foam Piece from FIG. 1—Number 2
7 3½″×¾″ Metal Strip centered from FIG. 1—Number 1
8 Display of Metal Strip Covered and Wrapped by Foam
9 Horizontal View of Foam Covering Metal
10 Horizontal View of Metal Covered by Foam
11 7″×1½″ Fabric Ribbon
12 Display of Metal Strip Covered by Foam, now also Wrapped by Fabric Ribbon
13 Metal Snap Button Attached to Final Product Created by FIG. 2 #12

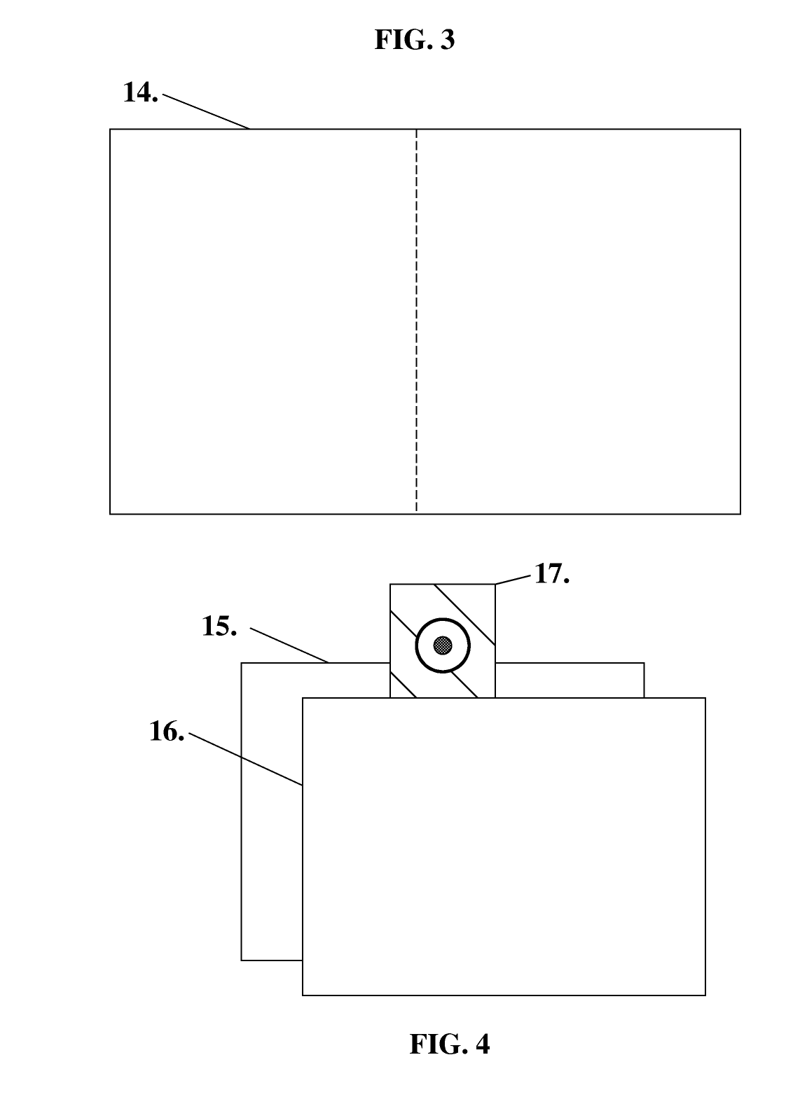

FIG. 3
14 2½″×3″ One Sided Self Adhesive Foam Piece

FIG. 4
15 Equal Part Resulting from Cutting Foam in FIG. 3 # 14 in Half
16 Equal Part Resulting from Cutting Foam in FIG. 3 # 14 in half
17 Frontal View of Inserted Metal Strip Created as Outlined in FIG. 2 # 12

FIG. 5
18 Rear View of Inserted Metal Strip Created as Outlined in FIG. 2 # 12

FIG. 6
19 FIG. 5 #18 product with the Metal Strip folded over, revealing the Final invention the “Heel Saver”

1. My invention is a device that gives a preventative solution to the blisters and rawness caused at the heel of your foot when shoes rub against your bare heel.
